(19) United States
(12) Patent Application Publication
(10) Pub. No.: US 2015/0335773 A1
(43) Pub. Date: Nov. 26, 2015

(54) METHODS OF IRRADIATING ARTICLES AND SANITIZING SYSTEMS EMPLOYING LIGHT DIFFUSING FIBERS

(71) Applicant: CORNING INCORPORATED, Corning, NY (US)

(72) Inventor: Anthony Bauco, Horseheads, NY (US)

(73) Assignee: CORNING INCORPORATED, Corning, NY (US)

(21) Appl. No.: 14/283,763

(22) Filed: May 21, 2014

(51) Int. Cl. A61L 2/10; A23B 7/015; G02B 6/02; A23L 3/28

(52) U.S. Cl. CPC A61L 2/10; A23L 3/28; A23B 7/015; G02B 6/02214

(57) ABSTRACT

A method of sanitizing a food article includes the steps: orienting a light-diffusing optical fiber in proximity to a food article; and directing ultraviolet light from an ultraviolet light source through a first end of the light-diffusing optical fiber. The food article is arranged on a substrate. The fiber comprises a glass composition and a plurality of scattering sites. The method also includes the steps: scattering the ultraviolet light off of the plurality of scattering sites and out of the fiber at about 0.1 dB/m to about 20 dB/m as scattered ultraviolet light; and sanitizing the food article with the scattered ultraviolet light.

### BACKGROUND

The disclosure generally relates to irradiating articles with ultraviolet light and, more particularly, to methods that employ light-diffusing optical fibers to sanitize food articles.

Ultraviolet (“UV”) light can be used to purify and/or decontaminate various articles, including food articles. For example, a UV lamp can be employed to sanitize food articles to kill or otherwise inhibit the growth of undesirable or dangerous bacteria and/or fungi. Sanitizing approaches and systems that employ such UV lamps, or equivalents, are limited in the sense that they emanate UV light from a single point source and only illuminate a relatively small area. Manufacturing throughput for a sanitizing process that relies on a point source, such as a UV lamp, will be limited to the particular area of the light source. Similarly, the power usage requirements of each lamp may also reduce the prospect of employing multiple UV lamps in a manufacturing sanitizing system or a residential unit such as a freezer.

Accordingly, there is a need for improved methods and systems for irradiating articles, such as food articles, that are: efficient in terms of power consumption, cost effective and efficacious.

### SUMMARY

According to one aspect, a method of irradiating an article is provided that includes the steps: orienting a light-diffusing optical fiber in proximity to an article; and directing ultraviolet light from an ultraviolet light source through a first end of the light-diffusing optical fiber. The fiber comprises a glass composition and a plurality of scattering sites. The method further includes the steps: scattering the ultraviolet light off of the plurality of scattering sites and out of the fiber as scattered ultraviolet light; and irradiating the article with the scattered ultraviolet light.

According to an additional aspect, a method of sanitizing a food article is provided that includes the steps: orienting a light-diffusing optical fiber in proximity to a food article; and directing ultraviolet light from an ultraviolet light source through a first end of the light-diffusing optical fiber. The food article is arranged on a substrate. The fiber comprises a glass composition and a plurality of scattering sites. The method also includes the steps: scattering the ultraviolet light off of the plurality of scattering sites and out of the fiber at about 0.1 dB/m to about 20 dB/m as scattered ultraviolet light; and sanitizing the food article with the scattered ultraviolet light.

According to a further aspect, a sanitizing system is provided that includes an article on a substrate; a light-diffusing optical fiber arranged in proximity to the substrate; and an ultraviolet light source configured to inject ultraviolet light into a first end of the optical fiber. The light-diffusing optical fiber includes a core region comprising fused silica having a plurality of scattering sites, and a cladding over the core region. The fiber is configured to (i) propagate the ultraviolet light along the fiber length, (ii) scatter the ultraviolet light at the plurality of scattering sites at about 0.1 dB/m to about 20 dB/m as scattered ultraviolet light, and (iii) emit the scattered ultraviolet light out of the cladding to irradiate the article.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiments, and together with the description serve to explain principles and operation of the various embodiments.

### BRIEF DESCRIPTION OF THE DRAWINGS

FIG. 1 is a schematic perspective view of a system for irradiating an article on a substrate having a light-diffusing optical fiber and an ultraviolet light source according to an aspect of the disclosure;

FIG. 1A is schematic perspective view of a system for irradiating an article on a substrate having a light-diffusing optical fiber, an ultraviolet light source and a visible light source according to a further aspect of the disclosure;

FIG. 1B is a schematic cross-sectional view of the fiber and substrate of the system for irradiating an article depicted in FIG. 1;

FIG. 1C is a schematic cross-sectional view of the fiber and substrate of the system for irradiating an article depicted in FIG. 1A;

FIG. 2 is a schematic of a sanitizing system employing an ultraviolet light source and light-diffusing fibers according to an additional aspect of the disclosure; and

FIG. 2A is a schematic of a sanitizing system employing an ultraviolet light source, a visible light source and light-diffusing fibers according to a further aspect of the disclosure.

### DETAILED DESCRIPTION

Reference will now be made in detail to the present preferred embodiments, examples of which are illustrated in the accompanying drawings. Whenever possible, the same reference numerals will be used throughout the drawings to refer to the same or like parts. It should be understood that the embodiments disclosed herein are merely examples, each incorporating certain benefits of the present disclosure.

Various modifications and alterations may be made to the following examples within the scope of the present disclosure, and aspects of different examples may be mixed in different ways to achieve yet further examples. Accordingly, the true scope of the disclosure is to be understood from the entirety of the present disclosure, in view of but not limited to the embodiments described herein.

Terms such as “horizontal,” “vertical,” “front,” “back,” etc., and the use of Cartesian Coordinates are for the sake of reference in the drawings and for ease of description and are not intended to be strictly limiting either in the description or in the claims as to an absolute orientation and/or direction.

In the description of the invention below, the following terms and phrases are used in connection to light-diffusing fibers.

The “refractive index profile” is the relationship between the refractive index or the relative refractive index and the waveguide (fiber) radius.

The “relative refractive index percent” is defined as:

Δ(r)%=100×[n(r)²−(n_{REF})²]/2n(r)²,

where n(r) is the refractive index at radius, r, unless otherwise specified. The relative refractive index percent Δ(r)% is defined at 850 nm unless otherwise specified. In one aspect, the reference index n_{REF} is silica glass with the refractive index of 1.452498 at 850 nm. In another aspect, n_{REF} is the maximum refractive index of the cladding glass at 850 nm. As used herein, the relative refractive index is represented by Δ and its values are given in units of “%”, unless otherwise specified. In cases where the refractive index of a region is less than the reference index n_{REF}, the relative index percent is negative and is referred to as having a depressed region or depressed-index, and the minimum relative refractive index is calculated at the point at which the relative index is most negative unless otherwise specified. In cases where the refractive index of a region is greater than the reference index n_{REF}, the relative index percent is positive and the region can be said to be raised or to have a positive index.

An “up-dopant” is herein considered to be a dopant which has a propensity to raise the refractive index of a region of a light-diffusing optical fiber relative to pure undoped SiO₂. A “down-dopant” is herein considered to be a dopant which has a propensity to lower the refractive index of a region of the fiber relative to pure undoped SiO₂. An up-dopant may be present in a region of a light-diffusing optical fiber having a negative relative refractive index when accompanied by one or more other dopants which are not up-dopants. Likewise, one or more other dopants which are not up-dopants may be present in a region of a light-diffusing optical fiber having a positive relative refractive index. A down-dopant may be present in a region of a light-diffusing optical fiber having a positive relative refractive index when accompanied by one or more other dopants which are not down-dopants.

Likewise, one or more other dopants which are not down-dopants may be present in a region of a light-diffusing optical fiber having a negative relative refractive index.

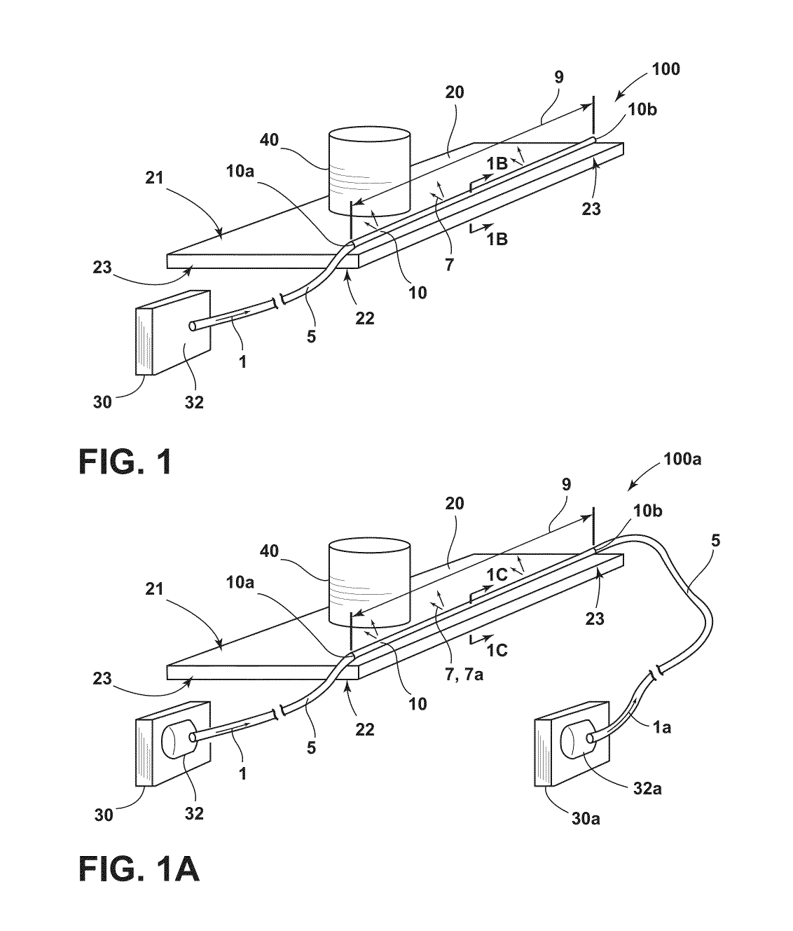

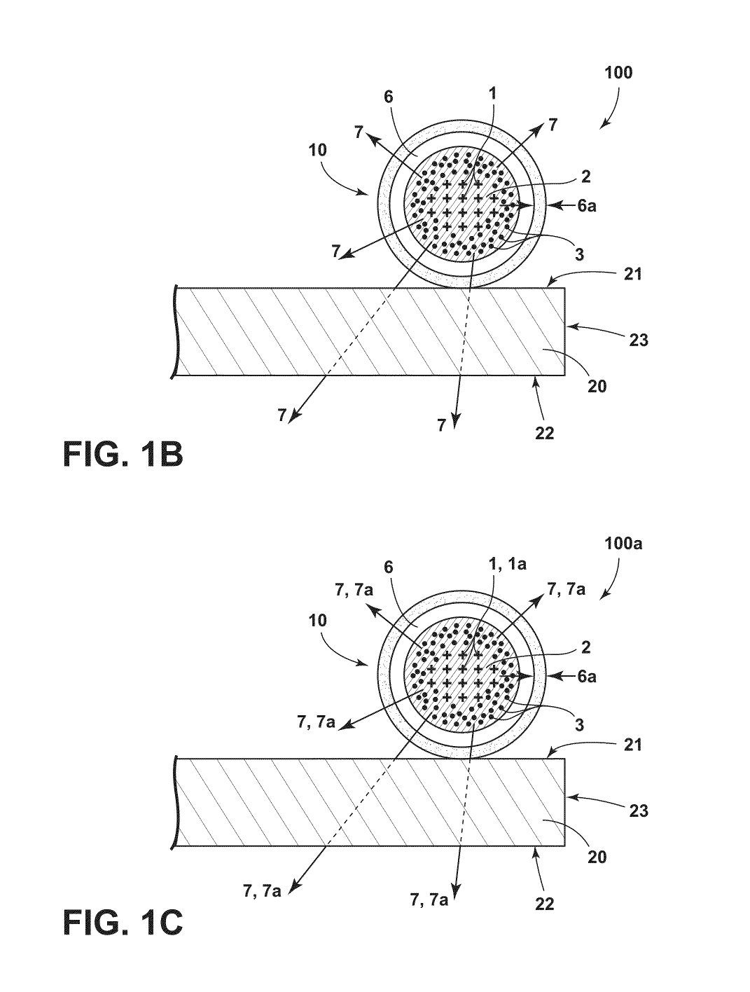

Referring to FIGS. 1 and 1B, a light-diffusing optical fiber 10, arranged as part of a system 100 for irradiating an article 40, is depicted according to one exemplary embodiment. In some aspects, the fiber 10 comprises a glass composition. The article 40 is arranged on or over a substrate 20. The substrate 20, as depicted in FIGS. 1 and 1B, includes a first primary surface 21, a second primary surface 22 and edges 23. The fiber 10 is arranged in proximity to the article 40. The fiber 10 includes a first end 10a and a second end 10b. The ends 10a and 10b define a length 9. Light-diffusing optical fiber 10 further includes a core region 2 and a cladding 6 over the core region 2. The optical fiber 10, as shown, can be connected to a UV light source 30 via a delivery fiber 5.

The core region 2 of the fiber 10 depicted in FIGS. 1 and 1B substantially comprises a fused silica glass composition with an index of refraction, n_{core}. In some embodiments, n_{core} is about 1.458. The core region 2 may have a radius ranging from about 20 μm to about 1500 μm. In some embodiments, the radius of the core region 2 is from about 30 μm to about 400 μm. In other embodiments, the radius of the core region 2 is from about 125 μm to about 300 μm. In still other embodiments, the radius of the core region 2 is from about 20 μm, 30 μm, 40 μm, 50 μm, 60 μm, 70 μm, 80 μm, 90 μm, 100 μm, 120 μm, 140 μm, 160 μm, 180 μm, 200 μm, 220 μm, 240 μm, 260 μm, 280 μm, 300 μm, 400 μm, 500 μm, 600 μm, 700 μm, 800 μm, 900 μm, 1000 μm, 1100 μm, 1200 μm, 1300 μm, 1400 μm or 1500 μm.

Still referring to FIGS. 1 and 1B, the core region 2 of the fiber 10 further includes a plurality of scattering sites 3. These scattering sites 3 are located in a scattering region within the core region 2 of light-diffusing optical fiber 10. These scattering sites 3 may comprise gas-filled voids or gaseous pockets (e.g. air-filled pockets), such as taught by U.S. application Ser. Nos. 12/950,045, 13/097,208, 13/269,055, and 13/713,224, herein incorporated by reference. In other embodiments, scattering sites 3 can comprise particles, such as micro- or nanoparticles of ceramic materials, configured to scatter UV light. It is preferable to select a medium for scattering sites 3 that demonstrates little absorption in the UV wavelengths (approximately 10 nm to 450 nm), for example, SiO₂ particles.

When gas-filled voids are employed for the plurality of scattering sites 3 in the core region 2, these voids may be distributed throughout the core region 2. The gas-filled voids employed as scattering sites 3 may also be located at the interface between core region 2 and the cladding 6, or they may be arranged in an annular ring within core region 2. The gas-filled voids may be arranged in a random or organized pattern and may run parallel to the length 9 of the fiber 10 or may be helical in shape (i.e., rotating along the long axis of the fiber 10 along the length 9). The scattering region within the core region 2 that contains the scattering sites 3 may comprise a large number of gas-filled voids, for example more than 50, more than 100, or more than 200 voids in the cross-section of the fiber 10. In other embodiments, the scattering sites 3 may comprise gas-filled voids at a volume fraction of about 0.1 to 30% in the core region 2. For embodiments of optical fiber 10 having a particularly long length, e.g., on the order of approximately 100 m, the volume fraction of gas-filled voids employed as scattering sites may approach zero to ensure sufficient propagation of light rays 1 down the length of the fiber without appreciable loss to the desired scattering locations. Further, in some embodiments, it is advantageous to vary the volume fraction of gas-filled voids as a function of fiber length to change the degree of light scattering at different locations of the fiber, depending on the application.

The gas-filled voids may contain, for example, SO₂, Kr, Ar, CO₂, N₂, O₂, or mixtures thereof. The cross-sectional size (e.g., approximate diameter) of the voids may be from about 1 nm to about 1 μm, or in some embodiments, the cross-sectional size may range from about 1 nm to about 10 μm. The length of each gas-filled void may vary from about 1 μm to about 100 m, and in some cases it may vary as a function of the overall length 9 of the fiber 10. In some embodiments, the cross-sectional size of the voids employed as scattering sites 3 is about 1 nm, 2 nm, 3, nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 120 nm, 140 nm, 160 nm, 180 nm, 200 nm, 250 nm, 300 nm, 400 nm, 500 nm, 600 nm, 700 nm, 800 nm, 1 μm, 2 μm, 3 μm, 4 μm, 5 μm, 6 μm, 7 μm, 8 μm, 9 μm, or 10 μm. In other embodiments, the length of the voids is about 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1000 μm, 5 mm, 10 mm, 50 mm, 100 mm, 500 mm, 1 m, 5 m, 10 m, 20m, 30m, 40m, 50 m, 60 m, 70 m, 80 m, 90 m, or 100 m.

The scattering sites 3 in the core region 2 of the light-diffusing optical fiber 10 are configured to scatter UV light rays 1 propagating within the core region 2 along the axial direction of the fiber 10. In particular, these sites 3 scatter the light rays 1 in substantially radial directions—i.e., as scattered UV light rays 7 outward from the core region 2, and through the cladding 6 of the fiber 10. These scattered UV light rays 7 illuminate the light-diffusing optical fiber 10 in the UV spectrum in the space surrounding the fiber 10. In turn, these scattered UV light rays 7 can be employed to kill bacteria and other microbes in proximity to the fiber 10, at least along the full length 9 of the fiber 10. As depicted in FIGS. 1 and 1B, the scattered UV light rays 7 generated by the system 100 can irradiate the article 40, as arranged over the substrate 20. Further, the scattered UV light rays 7 can kill bacteria and other microbes in article 40, e.g., when article 40 is a food item.

As also depicted in FIGS. 1 and 1B, the system 100 for irradiating the article 40 can include the UV light source 30 connected to the first end 10a of the light-diffusing optical fiber 10 by the delivery fiber 5. UV light source 30 can be employed to generate UV light rays 1 and direct the rays 1 into the delivery fiber 5. These UV light rays 1 are then directed from the delivery fiber 5 and into the first end 10a of the fibers 10. Suitable light sources for UV light source 30 include conventional high-brightness LED sources. The delivery fiber 5 can be a single fiber, a bundle of fibers or a single large étendue fiber that is subsequently spliced or coupled to a bundle of light diffusing fibers 10. Preferably, the delivery fiber 5 is configured to propagate UV light rays 1 without significant scattering and absorption at the UV wavelengths. In other embodiments, the UV light source 30 is directly connected to the first end 10a of the fibers 10, thereby eliminating the need for a delivery fiber.

The scatter-induced attenuation associated with voids employed as scattering sites 3 in the core region 2 of the fiber 10 may be increased by increasing the concentration of these voids, positioning the voids throughout the fiber 10, or in cases where the voids are limited to an annular ring-shaped region, by increasing the width of the annulus comprising the voids. In some embodiments, when the gas-filled voids employed as scattering sites 3 are helical in shape, the scattering-induced attenuation may also be increased by varying the pitch of the helical voids over the length of the fiber 10. Specifically, it has been found that helical voids with a smaller pitch scatter more light than helical voids with a larger pitch. Accordingly, the intensity of the illumination of the fiber 10 along its length 9 can be controlled (i.e., predetermined) by varying the pitch of the helical voids along the axial length 9. As used herein, the “pitch” of the helical voids refers to the inverse of the number of times the helical voids are wrapped or rotated around the long axis of the fiber 10 per unit length.

Referring again to FIGS. 1 and 1B, the light-diffusing optical fiber 10 further includes the cladding 6 arranged over the core region 2. The cladding 6 of fiber 10 can further comprise a polymer coating 6a, located over the outer surface of the cladding 6. As such, cladding 6 is preferably comprised of silica glass. It also preferable to employ a glass composition for cladding 6 with a low refractive index to increase the numerical aperture (“NA”) of the fiber 10. In some embodiments, the cladding 6 may comprise silica glass down-doped with fluorine, boron or a combination of these dopants. The NA of the fiber 10 may be from about 0.12 to about 0.30 for some embodiments, and may range from about 0.2 to about 0.3 for other embodiments. In other embodiments, the relative refractive index of the cladding may be less than −0.5%, and in still others less than −1%.

In light-diffusing optical fibers 10 employed in the system 100 for irradiating an article 40, the cladding 6 generally extends from the outer radius of the core region 2. In some embodiments, the thickness of the cladding 6 is greater than about 5 μm, greater than about 10 μm, greater than about 15 μm or greater than about 20 μm. In other embodiments, the cladding 6 has a thickness of about 5 μm, 10 μm, 15 μm, 20 μm, 25 μm, or 30 μm. In still other embodiments, the thickness of cladding 6 ranges from about 5 μm to about 30 μm.

For light-diffusing optical fibers 10, the overall fiber diameter (i.e., the diameter of core region 2 plus the thickness of cladding 6) ranges from about 125 μm to about 3000 μm. In further embodiments, the optical fibers 10 have an overall diameter that ranges from about 45 μm to about 3000 μm. In other embodiments, the optical fibers 10 have an overall diameter of about 45 μm, 50 μm, 60 μm, 70 μm, 80 μm, 90 μm, 100 μm, 125 μm, 150 μm, 175 μm, 200 μm, 225 μm, 250 μm, 275 μm, 300 μm, 350 μm, 400 μm, 450 μm, 500 μm, 600 μm, 700 μm, 800 μm, 900 μm, 1000 μm, 1500 μm, 2000 μm, 2500 μm, or 3000 μm.

Because light-diffusing optical fibers 10 operate in UV wavelengths, they can advantageously be utilized to kill bacteria and other microbes within or on article 40 and/or other objects arranged in proximity to the fiber 10. Preferably, light-diffusing fibers 10 are particularly configured to propagate UV light rays 1 at UV wavelengths.

In some embodiments described herein, the light-diffusing optical fibers 10 will generally have a length 9 from about 100 m to about 0.15 m. In some embodiments, the fibers 10 will generally have a length 9 of about 100 m, 75 m, 50 m, 40 m, 30 m, 20 m, 10 m, 9 m, 8 m, 7 m, 6 m, 5 m, 4 m, 3 m, 2 m, 1 m, 0.75 m, 0.5 m, 0.25 m, 0.15 m, or 0.1 m. Generally, the fibers 10 are tailored with a length 9 based on the dimensions of the substrate 20 and/or the article 40.

Further, the light-diffusing optical fibers 10 described herein have a scattering-induced attenuation loss of greater than about 0.1 dB/m and up to about 20 dB/m at UV wavelengths, including at a wavelength of 300 nm, 325 nm, 350 nm, 375 nm, 400 nm, 425 nm, or 450 nm. For example, in some embodiments, the scattering-induced attenuation loss may be greater than about 0.1 dB/m, 0.2 dB/m, 0.3 dB/m, 0.4 dB/m, 0.5 dB/m, 0.6 dB/m, 0.7 dB/m, 0.8 dB/m, 0.9 dB/m, 1 dB/m, 1.2 dB/m, 1.4 dB/m, 1.6 dB/m, 1.8 dB/m, 2.0 dB/m, 2.5 dB/m, 3.0 dB/m, 3.5 dB/m, 4 dB/m, 5 dB/m, 6 dB/m, 7 dB/m, 8 dB/m, 9 dB/m, 10 dB/m, or 20 dB/m at UV wavelengths including at a wavelength of 300 nm, 325 nm, 350 nm, 375 nm, 400 nm, 425 nm, or 450 nm.

As described herein, the light-diffusing optical fibers 10, depicted in FIGS. 1 and 1B, may be constructed to produce uniform illumination of UV light (e.g., scattered UV light rays 7) along the entire length 9 of the fiber 10, or uniform illumination along a segment of the fiber 10 which is less than its entire length 9. The phrase “uniform illumination,” as used herein, means that the intensity of light emitted from the fiber 10 does not vary by more than 25% over the specified length.

With regard to the light-diffusing fibers 10, the polymer coating 6a employed with or over the cladding 6 makes the fibers 10 particularly suitable for movement and insertion in various geometries, components and other features associated with the system 100 for irradiating an article 40. In particular, the polymer coating 6a gives the fibers 10 added flexibility and better lubricity for insertion or installation into various components of the system 100, including small diameter pipes, long substrates (e.g., substrate 20) and other features.

Referring again to FIGS. 1 and 1B, the light-diffusing optical fibers 10 further includes the cladding 6 arranged over the core region 2. In some embodiments, the cladding 6 may comprise silica glass down-doped with fluorine, boron or a combination of these dopants. In other embodiments, cladding 6 may comprise a polymeric composition. In some cases, the polymeric composition employed for cladding 6 is comparable to that employed for polymer coating 6a. When the cladding 6 comprises a polymeric composition, the NA of the fiber 10 may be greater than about 0.3 and up to about 0.5 for some embodiments, and may range from about 0.39 to about 0.53 for other embodiments. In other embodiments of fiber 10 having a cladding 6 comprising a polymeric composition, the relative refractive index of the cladding may be less than −0.5%, and in still others less than −1%. Conversely, when the cladding 6 comprises a glass composition, the NA of the fiber 20 may be from about 0.12 to about 0.30 for some embodiments, and may range from about 0.2 to about 0.3 for other embodiments. In other embodiments, the relative refractive index of the cladding 6 may be less than −0.5%, and in still others less than −1%.

As also depicted in FIGS. 1 and 1B, the polymer coating 6a employed with the light-diffusing optical fibers 10 of the system 100 may comprise a clear secondary coating layer that is comparable to the clear polymeric coatings typically employed in telecommunications optical fibers to facilitate mechanical handling. In some embodiments, polymer coating 6a is a layer coated on the outside surface of the cladding 6. In other embodiments, polymer coating 6a serves as the cladding 6 and is coated on the outside surface of core region 2. Such secondary coatings employed as a polymer coating 6a are described in U.S. application Ser. No. 13/713,224, herein incorporated by reference. For polymer coating 6a employed in light-diffusing optical fibers 10, the thickness of the coating 6a can be minimized to reduce the amount of UV light absorption. In certain embodiments, the composition of the polymer coating 6a is selected to minimize UV light absorption with light transmittance levels of 90% or greater. That is, the polymer coating 6a exhibits a light transmittance level of 90% or greater for UV light rays 1 and scattered UV light rays 7. In some embodiments, the polymer coating 6a can comprise an amorphous fluorinated polymer, such as DuPont™ Teflon® μF. In other embodiments, the polymer coating 6a can comprise an acrylate-based coating, such as CPC6, manufactured by DSM Desotech, Elgin, Ill. In some other embodiments, the polymer coating 6a can comprise a silicone-based polymer coating. In an additional set of embodiments, the polymer coating 6a can comprise a low refractive index polymeric material such as a UV- or thermally-curable fluoroacrylate, such as PC452 available from SSCP Co. Ltd., 403-2, Moknae, Ansan, Kyunggi, Korea.

In some embodiments of light-diffusing optical fibers 10, such as those depicted in connection with the system 100 in FIGS. 1 and 1B, the thickness of the polymer coating 6a can range from about 1 μm to about 15 μm. In some embodiments, the thickness of the polymer coating 6a ranges from about 0.1 μm to about 50 μm, including thickness values of 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, or 50 μm. Preferably, the thickness of the polymer coating 6a is set at a range from about 5 μm to about 10 μm.

Light-diffusing optical fiber 10 can be formed utilizing various techniques. For fiber embodiments in which the scattering sites 3 comprise gas-filled voids, these voids can be incorporated into the fibers by the methods described in U.S. application Ser. Nos. 11/583,098, 12/950,045, 13/097,208, 13/269,055, and 13/713,224, herein incorporated by reference. Generally, the light-diffusing optical fibers 10 are drawn from an optical fiber preform with a fiber take-up system and exit the draw furnace along a substantially vertical pathway (not shown). In some embodiments, fibers 10 are rotated as they are drawn to produce helical voids (serving as scattering sites 3) along the axial length 9. As the optical fiber 10 exits the draw furnace, a non-contact flaw detector may be used to examine the optical fiber for damage and/or flaws that may have occurred during the processing of the fibers. Thereafter, the diameter of the optical fiber 10 may be measured with a non-contact sensor. Optionally, the fiber 10 can be drawn through a cooling system which cools the optical fiber (not shown).

For light-diffusing optical fiber 10, the optional cooling step would be performed before the application of polymer coating 6a, and before the creation of cladding 6 when it comprises a polymeric composition. As the optical fiber 10 exits the cooling system, the fiber 10 enters at least one coating system where one or more polymer layers are applied to the cladding 6, thereby forming the polymer coating 6a. As the fibers 10 exit the polymer coating system, the diameter of the fibers can be measured using a non-contact sensor. Thereafter, a non-contact flaw detector can be used to examine the fibers 10 for damage and/or flaws in the cladding 6 and the polymer coating 6a that may have occurred during the manufacture of the fibers.

Referring again to FIGS. 1 and 1B, the system 100 can be configured such that its UV light source 30 is arranged with a dichroic mirror 32. The mirror 32, when present, is configured such that UV light rays 1 emanating from the light source 30 are directed through the mirror 32 before entering the delivery fiber 5. In this configuration, the dichroic mirror 32 would be configured to reflect light in the visible light spectrum, while allowing UV light rays 1 to pass. Such an arrangement can improve the efficiency of the system 100 in terms of ensuring UV light rays 1 are transmitted through the delivery fiber 5 and through the light-diffusing optical fiber 10 without significant loss and interference from light in the visible light spectrum.

One method for employing the system 100 to irradiate an article 40 depicted in FIGS. 1 and 1B includes the steps: (a) orienting the light-diffusing optical fiber 10 in proximity to the article 40; and (b) directing UV light rays 1 from a UV light source 30 through a first end 10a of the light-diffusing optical fiber 10. The fiber 10 comprises a glass composition and a plurality of scattering sites 3. The method of irradiating the article 40 further includes the steps: (c) scattering the UV light rays 1 off of the plurality of scattering sites 3 and out of the fiber 10 as scattered UV light rays 7; and (d) irradiating the article 40 with the scattered UV light rays 7. As such, the system 100 can be employed to direct scattered UV light rays 7 to irradiate the article 40. Depending on the composition of article 40 (e.g., as an object containing UV-curable polymer materials), its location relative to the fiber 10 and other considerations, the method can be used to treat the article 40 with the scattered UV light rays 7.

According to another aspect, another method for employing the system 100 to irradiate an article 40 depicted in FIGS. 1 and 1B includes the steps: (a) orienting the light-diffusing optical fiber 10 in proximity to the article 40; and (b) directing UV light rays 1 from a UV light source 30 through a first end 10a of the light-diffusing optical fiber 10. The fiber 10 comprises a glass composition and a plurality of scattering sites 3. Further, the article 40 is a food article in this configuration. This method of irradiating the article 40 further includes the steps: (c) scattering the UV light rays 1 off of the plurality of scattering sites 3 and out of the fiber 10 as scattered UV light rays 7; and (d) sanitizing the article 40, as a food article, with the scattered UV light rays 7. Further, the scattered UV light rays 7 are scattered out of the fiber 10 at about 0.1 dB/m to about 20 dB/m along the length 9.

According to another aspect, a system 100a for irradiating an article 40 is depicted in FIGS. 1A and 1C. In general, the system 100a includes a light-diffusing optical fiber 10, article 40 and substrate 20, each configured similarly to the like-numbered and named elements described in connection with system 100 (see FIGS. 1 and 1B). Further, all like-numbered and named elements shown in FIGS. 1A and 1C in connection with system 100a have the same or comparable structure and function the same or virtually the same as these same elements shown in FIGS. 1 and 1B in connection with system 100 and described in the foregoing.

The differences in system 100a depicted in FIGS. 1A and 1C relate to the addition of a visible light source 30a that is connected to the second end 10b of fiber 10 via a delivery fiber 5. As such, the visible light source 30a is arranged to generate visible light rays 1a that are directed into the delivery fiber 5. These light rays 1a are then propagated into the second end 10b of the fiber 10 and then through the fiber 10 along its length 9. A significant percentage of these light rays 1a are then scattered off of the plurality of scattering sites 3 and out of the fiber 10 as scattered visible light rays 7a. These scattered light rays 7a can then be used illuminate article 40.

In some aspects of the system 100a depicted in FIGS. 1A and 1C, the system 100a can be configured such that its visible light source 30a is arranged with a dichroic mirror 32a. The mirror 32a, when present, is configured such that visible light rays 1a emanating from the light source 30a are directed through the mirror 32a before entering the delivery fiber 5. In this configuration, the dichroic mirror 32a would be configured to reflect light in the non-visible light spectrum (e.g., infrared light, UV light, etc.), while allowing visible light rays 1a to pass. Such an arrangement can improve the efficiency of the system 100a in terms of ensuring visible light rays 1a are transmitted through the delivery fiber 5 and through the light-diffusing optical fiber 10 without significant loss and interference from light in the non-visible light spectrum.

According to a further aspect, a method for employing the system 100a to irradiate an article 40 depicted in FIGS. 1A and 1C includes the steps: (a) orienting the light-diffusing optical fiber 10 in proximity to the article 40; and (b) directing UV light rays 1 from a UV light source 30 through a first end 10a of the light-diffusing optical fiber 10. The fiber 10 comprises a glass composition and a plurality of scattering sites 3. Further, the article 40 may be a food article in this configuration. This method of irradiating the article 40 using the system 100a further includes the steps: (c) scattering the UV light rays 1 off of the plurality of scattering sites 3 and out of the fiber 10 as scattered UV light rays 7; and (d) sanitizing the article 40 (e.g., as a food article) with the scattered UV light rays 7. Further, the scattered UV light rays 7 are scattered out of the fiber 10 at about 0.1 dB/m to about 20 dB/m along the length 9. In addition, the method employing system 100a also includes the steps: (e) directing visible light rays 1a from the visible light source 30a into the second end 10b of the fiber 10; (f) scattering the visible light rays 1a off of the plurality of scattering sites 3 and out of the fiber 10 as scattered visible light rays 7a; and (g) illuminating the article 40 with the scattered visible light rays 7a.

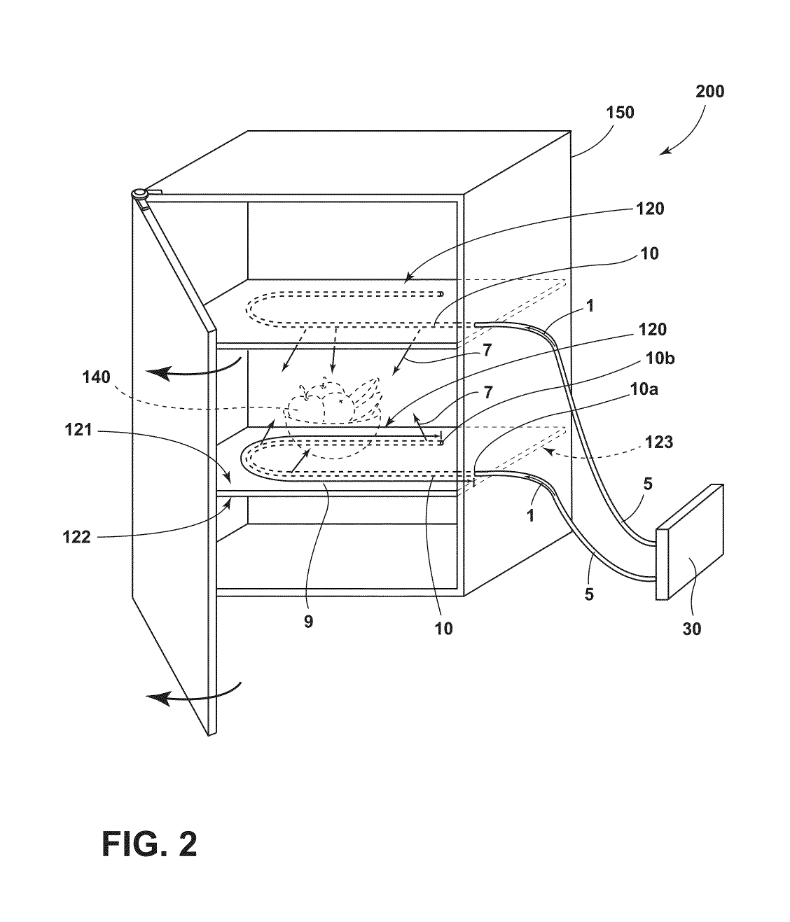

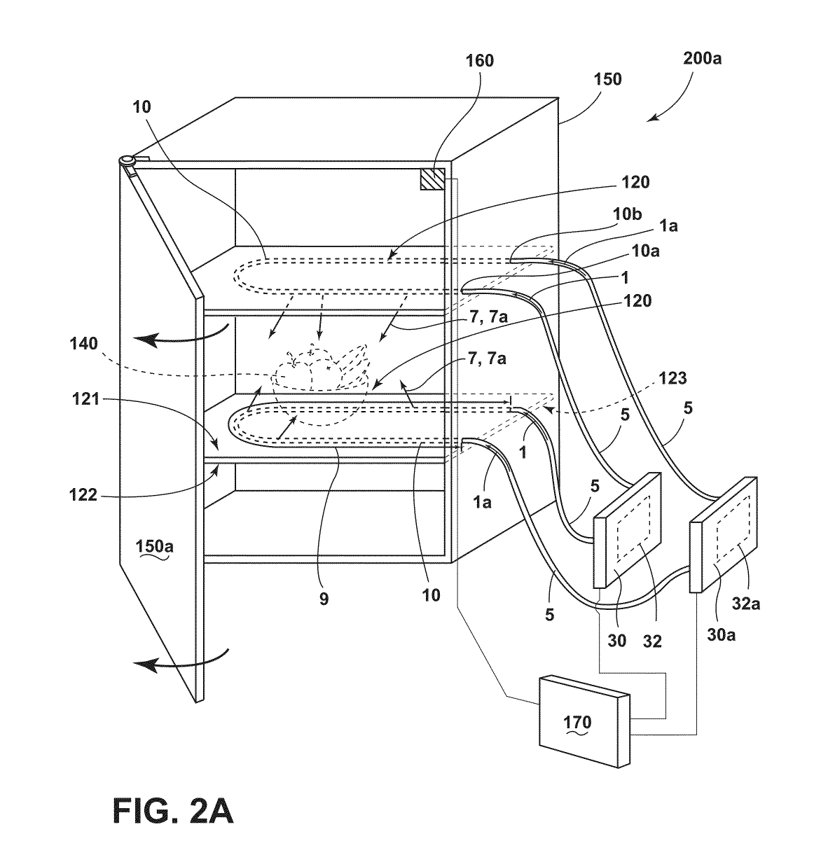

Referring to FIG. 2, a sanitizing system 200 that includes an article 140 on a substrate 120 within a cabinet 150 is depicted. Cabinet 150 may contain one or more substrates 120. Typically, article 140 is a food article. Further, the system 200 includes one or more light-diffusing optical fibers 10 arranged for irradiating the article 140. The light-diffusing fiber 10 depicted in FIG. 2 in connection with system 200 has the same structure and function as the fibers 10 described in the foregoing and depicted in FIGS. 1-1C. Other like-numbered and -named elements depicted in FIG. 2 also have the same or virtually the same structure and function within system 200 as described earlier in this disclosure.

Within the system 200 depicted in FIG. 2, the article 140 is arranged on or over the substrate 120. The substrate 120, as depicted in FIGS. 1 and 1B, includes a first primary surface 121, a second primary surface 122 and edges 123. The fiber 10 is arranged in proximity to the substrate 120. In some aspects, the fiber 10 includes a core region 2 comprising a fused silica glass composition having a plurality of scattering sites 3, and a cladding 6 over the core region 2.

Further, the fiber 10 employed in the system 200 depicted in FIG. 2 includes a first end 10a and a second end 10b. The ends 10a and 10b define a length 9. Light-diffusing optical fiber 10 further includes a core region 2 and a cladding 6 over the core region 2. Further, each optical fiber 10 can be connected to a UV light source 30 via a delivery fiber 5. The UV light source 30 is configured to inject UV light rays 1 into the first end 10a of each fiber 10 via a delivery fiber 5. The fiber 10 according to some aspects is configured to (i) propagate the UV light rays 1 along the fiber length 9, (ii) scatter the UV light rays 1 at the plurality of scattering sites 3 at about 0.1 dB/m to about 20 dB/m as scattered UV light rays 7, and (iii) emit the scattered UV light rays 7 out of the cladding 6 to irradiate the article 140. The system 200 for sanitizing the article 140 (e.g., a food article) has the advantage of being able to direct and place scattered UV light rays 7 throughout the cabinet 150 without the need for locating the UV light source 30 within the cabinet. Further, the fibers 10 can be snaked, positioned or otherwise arranged within the cabinet to maximize sanitizing efficiency while otherwise hiding or obscuring them from view within the cabinet 150.

In some aspects of system 200, the substrate 120 has a composition that is substantially transparent to the scattered UV light rays 7 emanating from the fibers 10. Preferably, the optical transmittance of the material selected for the substrate 120 exceeds 90%. As such, it is possible to arrange fibers 10, substrates 120 and article 140 in various configurations with cabinet 150 to maximize the irradiation and sanitizing efficiency of system 200 with regard to the article 140. Scattered UV light rays 7 can thus travel through a substrate 120 positioned between a fiber 10 and the article 140, and then through the cabinet 150 to assist in sanitizing the article 140.

According to another aspect, a system 200a for sanitizing an article 140 is depicted in FIG. 2A. In general, the system 200a includes one or more light-diffusing optical fibers 10, cabinet 150, article 140 and substrate 120, each configured similarly to the like-numbered and -named elements described in connection with system 200 (see FIG. 2). Further, all like-numbered and -named elements shown in FIG. 2A in connection with system 200a have the same or comparable structure and function the same or virtually the same as these same elements shown in FIG. 2 in connection with system 200 and described in the foregoing.

The differences in system 200a depicted in FIG. 2A relate to the addition of a visible light source 30a that is connected to the second end 10b of each fiber 10 via a delivery fiber 5. As such, the visible light source 30a is arranged to generate visible light rays 1a that are directed into the delivery fiber 5. These light rays 1a are then propagated into the second end 10b of the fiber 10 and then through the fiber 10 along its length 9. A significant percentage of these light rays 1a are then scattered off of the plurality of scattering sites 3 and out of the fiber 10 as scattered visible light rays 7a. These scattered light rays 7a can then be used illuminate article 140 within the cabinet 150.

In some aspects of the system 200a depicted in FIG. 2A, the system 200a can be configured such that its visible light source 30a is arranged with a dichroic mirror 32a. The mirror 32a, when present, is configured such that visible light rays 1a emanating from the light source 30a are directed through the mirror 32a before entering the delivery fiber 5. In this configuration, the dichroic mirror 32a would be configured to reflect light in the non-visible light spectrum, while allowing visible light rays 1a to pass. This type of an arrangement can improve the efficiency of the system 200a in terms of ensuring visible light rays 1a are transmitted through the delivery fiber 5 and through the light-diffusing optical fiber 10 without significant loss and interference from light in the non-visible light spectrum.

According to a further aspect, a method for employing the system 200a to sanitize an article 140 depicted in FIG. 2A includes the steps: (a) orienting the light-diffusing optical fibers 10 in proximity to the article 140 within the cabinet 150; and (b) directing UV light rays 1 from a UV light source 30 through a first end 10a of the light-diffusing optical fibers 10. The fibers 10 can each comprise a glass composition and a plurality of scattering sites 3. Further, the article 140 may be a food article in this configuration.

This method of sanitizing the article 140 (e.g., a food article) using the system 200a can further include the steps: (c) scattering the UV light rays 1 off of the plurality of scattering sites 3 and out of the fiber 10 as scattered UV light rays 7; and (d) sanitizing the article 140 with the scattered UV light rays 7. Further, the scattered UV light rays 7 are scattered out of the fiber 10 at about 0.1 dB/m to about 20 dB/m along the length 9. In addition, the method employing system 200a also includes the steps: (e) directing visible light rays 1a from the visible light source 30a into the second end of the fiber 10b for each of the fibers 10 configured within the cabinet 150; (f) scattering the visible light rays 1a off of the plurality of scattering sites 3 and out of the fibers 10 as scattered visible light rays 7a; and (g) illuminating the article 140 within the cabinet 150 with the scattered visible light rays 7a. In some aspects, the visible light source 30a is configured to propagate the visible light rays 1a along the fiber length 9; scatter the visible light rays 1a at the plurality of scattering sites 3 at about 0.1 dB/m to about 20 dB/m as scattered visible light rays 7a; and emit the scattered visible light rays 7a out of the cladding 6 to illuminate the article 140.

In some aspects, the system 200a depicted in FIG. 2A is further configured with a controller 170 to control the operation of light sources 30 and 30a to inject UV light rays 1 and visible light rays 1a into the fibers 10. As such, controller 170 is coupled to the light sources 30 and 30a. Further, a sensor 160 is arranged within the cabinet 150 to detect the motion of cabinet door 150a. The sensor 160 is coupled to or is otherwise in communication with controller 170 to provide a digital or analog input corresponding to the motion or state of door 150a. According to one aspect, controller 170 can be used or otherwise programmed to deactivate UV light source 30 and activate visible light source 30a upon detecting via input from sensor 160 an “open” state of door 150a with regard to the cabinet 150. As such, the article 140 can be illuminated with scattered visible light rays 7a without risk of imparting potentially hazardous scattered UV light rays 7 upon an individual positioned in front of the cabinet 150 with the door 150a in such an “open” state. On the other hand, controller 170 can be employed to activate UV light source 30 and deactivate visible light source 30a upon detection of a “closed” state of door 150a with regard to the cabinet 150. In this state, the article 140 can be sanitized by the scattered UV light rays 7 without the need for illumination via scattered visible light rays 7a. As such, energy associated with the operation of visible light source 30a can be conserved for efficient operation of the system 200a.

It will be apparent to those skilled in the art that various modifications and variations can be made without departing from the spirit or scope of the claims.

What is claimed is:

1. A method of irradiating an article, comprising the steps:
orienting a light-diffusing optical fiber in proximity to an article;
directing ultraviolet light from an ultraviolet light source through a first end of the light-diffusing optical fiber, the fiber comprising a glass composition and a plurality of scattering sites;
scattering the ultraviolet light off of the plurality of scattering sites and out of the fiber as scattered ultraviolet light; and
irradiating the article with the scattered ultraviolet light.

2. The method according to claim 1, wherein the fiber further comprises a second end, the ends defining a fiber length; and a core region along the fiber length that comprises silica and the plurality of scattering sites.

3. The method according to claim 2, wherein the fiber further comprises a cladding along the fiber length having an optical transmittance of 90% or greater for the scattered ultraviolet light.

4. The method according to claim 2, wherein the ultraviolet light has a wavelength from about 10 nm to about 450 nm.

5. The method according to claim 2, wherein the ultraviolet light has a wavelength from about 300 nm to about 450 nm.

6. The method according to claim 2, wherein the scattering step comprises scattering the ultraviolet light at about 0.1 dB/m to about 20 dB/m off of the plurality of scattering sites.

7. The method according to claim 2, wherein the plurality of scattering sites consist essentially of randomly oriented air pockets.

8. A method of sanitizing a food article, comprising the steps:
orienting a light-diffusing optical fiber in proximity to a food article, the food article arranged on a substrate;
directing ultraviolet light from an ultraviolet light source through a first end of the light-diffusing optical fiber, the fiber comprising a glass composition and a plurality of scattering sites;
scattering the ultraviolet light off of the plurality of scattering sites and out of the fiber at about 0.1 dB/m to about 20 dB/m as scattered ultraviolet light; and
sanitizing the food article with the scattered ultraviolet light.

9. The method according to claim 8, wherein the fiber further comprises a second end, the ends defining a fiber length; and a core region along the fiber length that comprises silica and the plurality of scattering sites.

10. The method according to claim 9, wherein the fiber further comprises a cladding along the fiber length having an optical transmittance of 90% or greater for the scattered ultraviolet light.

11. The method according to claim 9, wherein the ultraviolet light has a wavelength from about 10 nm to about 450 nm.

12. The method according to claim 9, wherein the ultraviolet light has a wavelength from about 300 nm to about 450 nm.

13. The method according to claim 9, wherein the plurality of scattering sites consist essentially of randomly oriented air pockets.

14. The method according to claim 9, further comprising the steps:
directing visible light from a visible light source into the second end of the fiber;
scattering visible light off of the plurality of scattering sites and out of the fiber as scattered visible light; and
illuminating the food article with the scattered visible light.

15. A sanitizing system, comprising:
an article on a substrate;
a light-diffusing optical fiber arranged in proximity to the substrate; and
an ultraviolet light source configured to inject ultraviolet light into a first end of the optical fiber, wherein the light-diffusing optical fiber comprises:
(a) a core region comprising fused silica having a plurality of scattering sites, and
(b) a cladding over the core region, and
further wherein the fiber is configured to (i) propagate the ultraviolet light along the fiber length, (ii) scatter the ultraviolet light at the plurality of scattering sites at about 0.1 dB/m to about 20 dB/m as scattered ultraviolet light, and (iii) emit the scattered ultraviolet light out of the cladding to irradiate the article.

16. The system according to claim 15, wherein the plurality of scattering sites consist essentially of randomly oriented air pockets.

17. The system according to claim 15, wherein the cladding has an optical transmittance of 90% or greater for the scattered ultraviolet light.

18. The system according to claim 15, wherein the ultraviolet light has a wavelength from about 10 nm to about 450 nm.

19. The system according to claim 15, wherein the ultraviolet light has a wavelength from about 300 nm to about 450 nm.

20. The system according to claim 15, wherein the plurality of scattering sites consist essentially of randomly oriented air pockets.

21. The system according to claim 15, further comprising:
a visible light source configured to inject visible light into a second end of the optical fiber, and wherein the fiber is further configured to (iv) propagate the visible light along the fiber length, (v) scatter the visible light at the plurality of scattering sites at about 0.1 dB/m to about 20 dB/m as scattered visible light, and (vi) emit the scattered visible light out of the cladding to illuminate the article.

22. The system according to claim 21, wherein the ultraviolet light source is configured with a first dichroic mirror, and the visible light source is configured with a second dichroic mirror, and further wherein the first mirror is arranged to substantially reflect visible light and the second mirror is arranged to substantially reflect ultraviolet light.
